(19) United States
(12) Patent Application Publication
(10) Pub. No.: US 2026/0038644 A1
(43) Pub. Date: Feb. 5, 2026

(54) MEMORY DEVICE, NETWORK SWITCH, AND DATA PROCESSING SYSTEM FOR PERFORMING MULTI LAYER SUMMATION OPERATION

(71) Applicant: Panmnesia Inc., Daejeon (KR)

(72) Inventor: Mi Ryeong KWON, Daejeon (KR)

(73) Assignee: Panmnesia Inc., Daejeon (KR)

(21) Appl. No.: 18/928,152

(22) Filed: Oct. 27, 2024

(30) Foreign Application Priority Data
Jul. 31, 2024 (KR) 10-2024-0102050

(51) Int. Cl. G16C 20/20; B82Y 35/00

(52) U.S. Cl. CPC G16C 20/20; B82Y 35/00

(57) ABSTRACT

A data processing system configured to calculate a reference particle force according to embodiments, includes a plurality of memory devices configured to generate a plurality of first grid point information and calculate at least one of a plurality of proximity forces, a plurality of first network switches configured to generate the plurality of second grid point information and calculate a plurality of first grid forces, a second network switch configured to generate third grid point information and calculates a plurality of second grid forces, and a host device configured to receive the third grid point information and calculate the reference particle force.

### CROSS-REFERENCE TO RELATED APPLICATION

This application is based on and claims priority under 35 U.S.C. § 119 to Korean Patent Application No. 10-2024-0102050, filed on Jul. 31, 2024, in the Korean Intellectual Property Office, the disclosure of which is incorporated by reference herein in its entirety.

### BACKGROUND

### 1. Field

The disclosure relates to a memory device, a network switch, and a data processing system capable of performing multi-layer summation operations.

### 2. Description of the Related Art

When developing new drugs, new materials, etc., calculations of forces between all atoms within a substance may be required to predict the properties of a medicine or material. In this way, as a method of calculating the forces for all atom-atom pairs included in a substance, a multi-layer summation operation method may be used. In the multi-layer summation operation method, force operations are processed between multiple atoms in parallel. Thus, there is a need for developing a method of efficiently performing multi-layer summation operations.

### SUMMARY

Provided are a host memory device, a network switch, and a data processing system capable of efficiently perform multi-layer summation operations.

Additional aspects will be set forth in part in the description which follows and, in part, will be apparent from the description, or may be learned by practice of the presented embodiments of the disclosure.

According to an aspect of the disclosure, a memory device includes a memory storing particle data about a reference particle and a plurality of associated particles and a processor configured to generate grid point information by replacing the plurality of associated particles with grid points based on the particle data and calculate at least one of a plurality of proximity forces defined as forces between a plurality of adjacent associated particles located within a reference distance from the reference particle among the plurality of associated particles and the reference particle.

The particle data according to an aspect of the disclosure may include at least one of position information of particles included in a target material, velocity information of the particles, energy information of the particles, mass information of the particles, and a list of nearby neighbors of the particles.

The processor according to an aspect of the disclosure may further be configured to generate the grid point information based on the energy information of the plurality of associated particles included in the particle data.

the grid point information according to an aspect of the disclosure may include location information of the grid point and energy information of the grid point.

The memory device according to an aspect of the disclosure may further include a communication circuit configured to transmit one or more proximity forces calculated by the processor and the grid point information to one of a plurality of network switches.

According to an aspect of the disclosure, a network switch includes a communication circuit configured to receive a plurality of input grid point information from a plurality of memory devices or a plurality of lower-level network switches and a processor configured to generate output grid point information by calculating a plurality of grid forces defined as forces between each of a plurality of input grid points corresponding to the plurality of input grid point information and a reference particle.

The input grid point information according to an aspect of the disclosure may include location information of the input grid point and energy information of the input grid point.

The network switch according to an aspect of the disclosure may further include a memory storing a lookup table for storing results of operations used in the calculation of the plurality of grid forces, wherein the processor may include an accelerator configured to calculate the plurality of grid forces using the lookup table.

The output grid point information according to an aspect of the disclosure may include position information of the output grid point determined based on the plurality of grid forces, and energy information of the output grid point.

the communication circuit according to an aspect of the disclosure may further be configured to transmit the output grid point information to either a higher-level network device or a host device.

According to an aspect of the disclosure, a data processing system configured to calculate a reference particle force defined as a force between a reference particle and a plurality of associated particles, and performs communication using a Computer Express Link (CXL), the data processing system includes a plurality of memory devices configured to generate a plurality of first grid point information by replacing the plurality of associated particles with first grid points based on particle data about a reference particle and a plurality of associated particles, and calculate at least one of a plurality of proximity forces defined as forces between a plurality of adjacent associated particles located within a reference distance from the reference particle among the plurality of associated particles and a reference particle, a plurality of first network switches configured to generate the plurality of second grid point information by receiving at least some of the plurality of first grid point information and calculating a plurality of first grid forces defined as forces between each of a plurality of first grid points corresponding to at least some of the plurality of first grid point information and a reference particle, a second network switch configured to generate third grid point information by receiving the plurality of second grid point information and calculates a plurality of second grid forces defined as forces between each of the plurality of second grid points corresponding to the plurality of second grid point information and a reference particle, and a host device configured to receive the third grid point information and calculate the reference particle force based on the third grid point information.

Each of the plurality of first grid point information of the data processing system according to an aspect of the disclosure may include position information of each of the plurality of first grid points and energy information of each of the plurality of first grid points, each of the plurality of second grid point information includes position information of each of the plurality of second grid points and energy information of each of the plurality of second grid points, and the third grid point information includes position information of the third grid point and energy information of the third grid point.

The plurality of first network switches of the data processing system according to an aspect of the disclosure may store a first lookup table that stores results of operations used in the calculation of the plurality of first grid forces, and are configured to calculate the plurality of first grid forces using the first lookup table, wherein the second network switch may be configured to store a second lookup table that stores results of operations used in the calculation of the plurality of second grid forces, and calculate the plurality of second grid forces using the second lookup table.

Each of the plurality of first network switches of the data processing system according to an aspect of the disclosure may be configured to generate the plurality of second grid point information of a first time point by calculating, at the first time point, at least one of the plurality of proximity forces of the first time point by each of the plurality of memory devices, and calculate the plurality of first grid forces of the first time point based on at least some of the plurality of first grid point information at a second time point defined as a time point after a reference time has elapsed from the first time point.

The second network switch of the data processing system according to an aspect of the disclosure may further be configured to generate the third grid point information of the first time point by calculating the plurality of second grid forces of the first time point based on the plurality of second grid point information of the first time point at a third time point that is a time point after the reference time has elapsed from the second time point.

Each of the plurality of first network switches of the data processing system according to an aspect of the disclosure may further be configured to generate the plurality of second grid point information of the first time point by calculating the plurality of first grid forces of the first time based on at least some of the plurality of first grid point information when at least one of the plurality of proximity forces at the second time is calculated by each of the plurality of memory devices.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a data processing system according to an embodiment;

FIG. 2 is a diagram of an example of a connection structure of a data processing system according to an embodiment;

FIG. 3 is a block diagram of a memory device according to an embodiment;

FIG. 4 is a diagram for explaining a method of calculating a plurality of proximity forces in a memory device according to an embodiment;

FIG. 5 is a diagram for explaining grid point information generated in a memory device according to an embodiment;

FIG. 6 is a block diagram of a network switch according to an embodiment;

FIGS. 7A to 7D are diagrams for explaining a method of calculating a plurality of first grid forces in each of a plurality of first network switches according to an embodiment; and

FIG. 8 is a diagram for explaining a method of calculating a second grid force in a second network switch according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the disclosure will be described in detail with reference to the accompanying drawings.

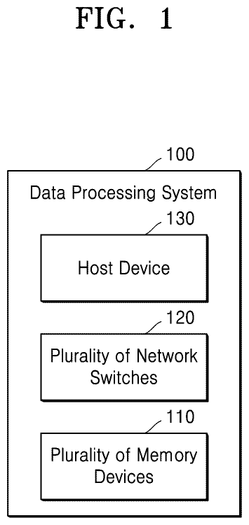

FIG. 1 is a block diagram of a data processing system 100 according to an embodiment.

Referring to FIG. 1, the data processing system 100 according to an embodiment may include a plurality of memory devices 110, a plurality of network switches 120, and a host device 130.

The data processing system 100 may be a system for calculating a reference particle force in a target material.

The target material may include a reference particle and at least some of a plurality of associated particles. The reference particle may be a particle that is the center of the reference particle force calculation. The plurality of associated particles may include particles other than the reference particle among particles included in the target material. In addition, the plurality of associated particles may include particles that are not included in the target material but are located around the target material and affect the characteristics of the target material. Here, the particle may be any one of an atom, a proton, a neutron, an electron, etc.

The reference particle force may be a force between the reference particle and a plurality of associated particles. For example, if the plurality of associated particles include first to m^{th} associated particles, the reference particle force may be calculated by adding up all of forces between the reference particle and the first associated particle, a force between the reference particle and the second associated particle, . . . , and a force between the reference particle and the m^{th} associated particle. In this case, if calculating the reference particle force by adding up all of the forces between the reference particle and each of the plurality of associated particles, data for all particles must be used, so a large amount of data transmission and reception is required, which increases the communication overhead, and a large number of calculations may be required, which may increase the calculation time. In order to reduce the communication overhead and the calculation time, the reference particle force may be calculated by using a multi-layer summation calculation method.

The multi-layer summation operation method may be a method of calculating the reference particle force by calculating a plurality of proximity forces defined as forces between a plurality of proximity associated particles and a reference particle, generating grid point information by replacing the plurality of associated particles with grid points, calculating the force between atoms and grid points based on grid point information, and repeating the calculation while increasing a spacing between grid points.

Such a multi-layer summation calculation method may be used for predicting the characteristics of new drugs in new drug development, predicting the physical properties and characteristics of materials in new material development, analyzing devices, materials, and living organisms in the aerospace field, and calculating particles at each point in the earth science field.

The plurality of memory devices 110, the plurality of network switches 120, and the host device 130 included in the data processing system 100 may communicate using a communication protocol such as InfiniBand, Ethernet, Peripheral Component Interconnect express (PCle), or Computer Express Link (CXL).

The plurality of memory devices 110 may be devices that store various types of data. For example, each of the plurality of memory devices 110 may be a device including any one of a solid-state drive (SSD), a dynamic random access memory (DRAM), a storage class memory (SCM), etc.

In an example embodiment, each of the plurality of memory devices 110 may store particle data regarding a reference particle and a plurality of associated particles.

In an example embodiment, each of the plurality of memory devices 110 may generate grid point information by replacing a plurality of associated particles with grid points based on particle data. Each of the plurality of memory devices 110 may generate grid point information based on energy information of a plurality of associated particles included in the particle data. At this time, the energy information may include information necessary for energy calculation of an actual particle, such as positions and charges of the plurality of associated particles.

In an example embodiment, each of the plurality of memory devices 110 may calculate one or more of a plurality of proximity forces, which are forces between a plurality of proximity associated particles located within a reference distance from a reference particle among the plurality of associated particles and the reference particle. The plurality of proximity associated particles may be associated particles located within a reference distance from a reference particle among the plurality of associated particles.

Each of the plurality of memory devices 110 may transmit one or more calculated proximity forces and grid point information to one of the plurality of network switches 120.

The plurality of network switches 120 may connect between the plurality of memory devices 110 and the host device 130. the plurality of network switches 120 may be hierarchically connected between the plurality of memory devices 110 and a host device 130.

In an example embodiment, each of the plurality of network switches 120 may receive a plurality of input grid point information from the plurality of memory devices 110 or a plurality of lower-level network switches. The lower-level network switch may denote a network switch that is connected to the plurality of memory devices 110 through a smaller number of network switches than its connections. In addition, the input grid point information may denote grid point information input to itself. The input grid point information may include position information of the input grid point and energy information of the input grid point.

In an example embodiment, each of the plurality of network switches 120 may generate output grid point information by calculating a plurality of grid forces, which are forces between each of the plurality of grid points corresponding to the plurality of input grid point information and the reference particle. The output grid point information may refer to grid point information output by the output grid point.

In an example embodiment, when performing calculations of the plurality of grid forces using a memoization method, each of the plurality of network switches 120 may store a lookup table that stores results of calculations used in calculating the plurality of grid forces. When performing calculations of the plurality of grid forces using a memoization method, each of the plurality of network switches 120 may calculate the plurality of grid forces using the lookup table.

The output grid point information may include position information of output grid points determined based on the plurality of grid forces and energy information of the output grid points.

In an example embodiment, each of the plurality of network switches 120 may transmit the output grid point information to one of an upper-level network devices and the host device 130. Here, the upper-level network switch may refer to a network switch that is connected to the host device 130 through a smaller number of network switches than itself.

The host device 130 may be a device that performs a calculation required in the data processing system 100. For example, the host device 130 may be any one of a Central Processing Unit (CPU), a Graphic Processing Unit (GPU), an Neural Processing Unit (NPU), etc.

The host device 130 may receive grid point information from a top-level network switch. The top-level network switch may denote a network switch directly connected to the host device 130.

In an example embodiment, the host device 130 may calculate a reference particle force based on the grid point information.

If the data processing system 100 according to an embodiment is used, the reference particle force calculation may be performed at a faster speed by calculating a plurality of proximity forces in the plurality of memory devices 110, calculating a plurality of grid forces in the plurality of network switches 120, and calculating a reference particle force in the host device 130.

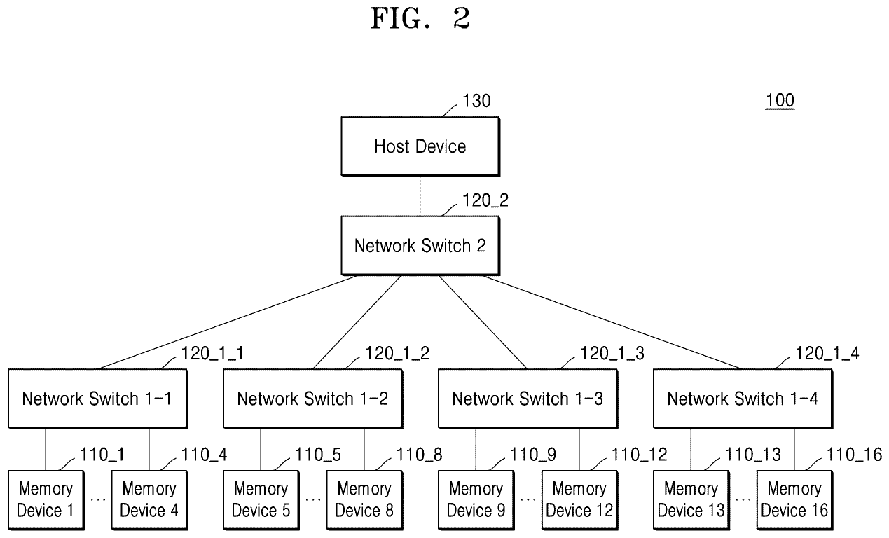

FIG. 2 is a drawing showing an example of a connection structure of the data processing system 100 according to an embodiment.

Referring to FIG. 2, the data processing system 100 according to an embodiment may include a plurality of memory devices 110_1 to 110_16, a plurality of network switches 120_1_1 to 120_1_4, and 120_2, and the host device 130.

In the embodiment of FIG. 2, the plurality of memory devices 110_1 to 110_16 may include the first memory device 110_1 to the sixteenth memory devices 110_16. The plurality of network switches 120_1_1 to 120_1_4, and 120_2 may include the plurality of first network switches 120_1_1 to 120_1_4 and the second network switch 120_2.

In the embodiment of FIG. 2, the plurality of first network switches 120_1_1 to 120_1_4 are directly connected to the plurality of memory devices 110_1 to 110_16, and thus may be referred to as the lowest level network switches. Conversely, the second network switch 120_2 is directly connected to the host device 130, and thus may be referred to as the highest level network switch. At this time, the plurality of first network switches 120_1_1 to 120_1_4 may be lower-level network switches than the second network switch 120_2, and the second network switch 120_2 may be higher-level network switches than the plurality of first network switches 120_1_1 to 120_1 4. In the embodiment of FIG. 2 described above, a plurality of network switches 120_1 1 to 120_1_4, 120_2 are configured in two levels, but the embodiment is not limited thereto. Thus, unlike the embodiment of FIG. 2, three or more levels may be configured. However, the following description will be mainly focused on the embodiment of FIG. 2.

In addition, although the embodiment of FIG. 2 illustrates an embodiment in which four memory devices or four lower-level network switches are connected to one network switch, the embodiment is not limited thereto. In the case when a plurality of associated particles are replaced with a square-shaped grid, the plurality of memory devices 110_1 to 110_16 and a plurality of network switches 120_1_1 to 120_1_4, and 120_2 may be connected as in the embodiment of FIG. 2. In contrast, in the case when a plurality of associated particles are replaced with a grid of a different shape, unlike the embodiment of FIG. 2, m memory devices (m is the number of grid points included in one grid depending on the shape of the grid) or m lower-level network switches may be connected to one network switch.

Each of the plurality of memory devices 110_1 to 110_16 may be connected to one of the plurality of first network switches 120_1_1 to 120_1_4. In the embodiment of FIG. 2, the first to fourth memory devices 110_1 to 110_4 may be connected to the 1-1 network switch 120_1_1, the fifth to eighth memory devices 110_5 to 110_8 may be connected to the 1-2 network switch 120_1_2, the ninth to twelfth memory devices 110_9 to 110_12 may be connected to the 1-3 network switch 120_1_3, and the thirteenth to sixteenth memory devices 110_13 to 110_16 may be connected to the 1-4 network switch 120_1_4.

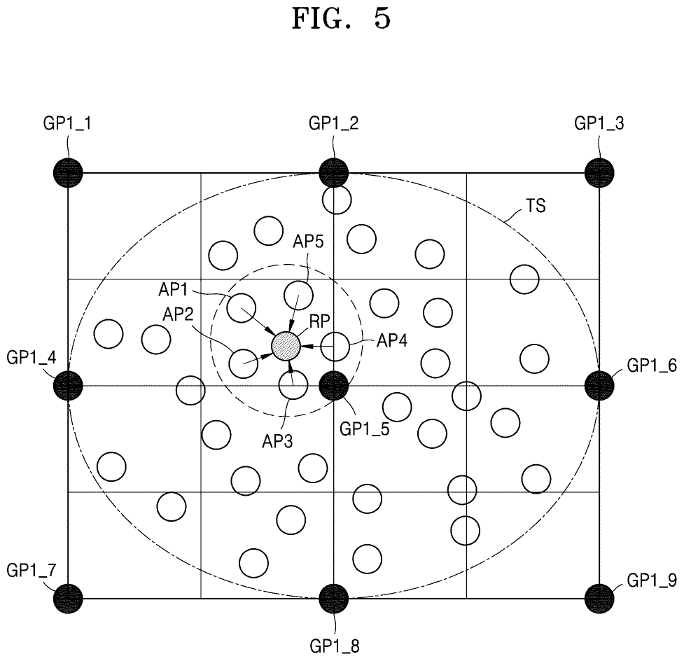

Each of the plurality of memory devices 110_1 to 110_16 may generate a plurality of first grid point information by replacing the plurality of associated particles with grid points based on particle data regarding the reference particle and the plurality of associated particles. Each of the plurality of first grid point information may include position information of each of the plurality of first grid points and energy information of each of the plurality of first grid points. At this time, an example in which the plurality of memory devices 110_1 to 110_16 generate each of the plurality of first grid point information will be described below with reference to FIG. 5.

In the embodiment of FIG. 2, the k^{th} memory device 110_k (where k is a natural number 1 to 16) may generate 1-k^{th} grid point information and transmit it to a connected network switch among the plurality of first network switches 120_1_1 to 120_1 4. The plurality of first network switches 120_1_1 to 120_1_4 may receive at least some of the plurality of first grid point information as input grid point information.

In addition, each of the plurality of memory devices 110_1 to 110_16 may calculate one or more of the plurality of proximity forces, which are forces between the reference particle and the plurality of proximity associated particles located within a reference distance among the plurality of associated particles. Each of the plurality of memory devices 110_1 to 110_16 may calculate one or more of the plurality of proximity forces using a formula that is commonly used for calculating forces between particles.

At this time, each of the plurality of memory devices 110_1 to 110_16 may calculate different proximity forces from different memory devices. For example, the first memory device 110_1 may calculate the proximity force between the reference particle and the first proximity associated particle, and the second memory device 110_1 may calculate the proximity force between the reference particle and the second proximity associated particle.

Each of the plurality of memory devices 110_1 to 110_16 may transmit a plurality of proximity forces to the host device 130 through the plurality of network switches 120_1_1 to 120_1_4, and 120_2.

Each of the plurality of first network switches 120_1_1 to 120_1_4 may receive at least some of the plurality of first grid point information. Each of the plurality of first network switches 120_1_1 to 120_1_4 may generate a plurality of second grid point information by calculating a plurality of first grid forces, which are forces between the plurality of first grid points corresponding to at least some of the plurality of first grid point information and the reference particle. When performing calculations of the plurality of grid forces using the memoization method, each of the plurality of first network switches 120_1_1 to 120_1_4 may store a first lookup table that stores the results of calculations used for calculating the plurality of first grid forces and may calculate the plurality of first grid forces using the first lookup table.

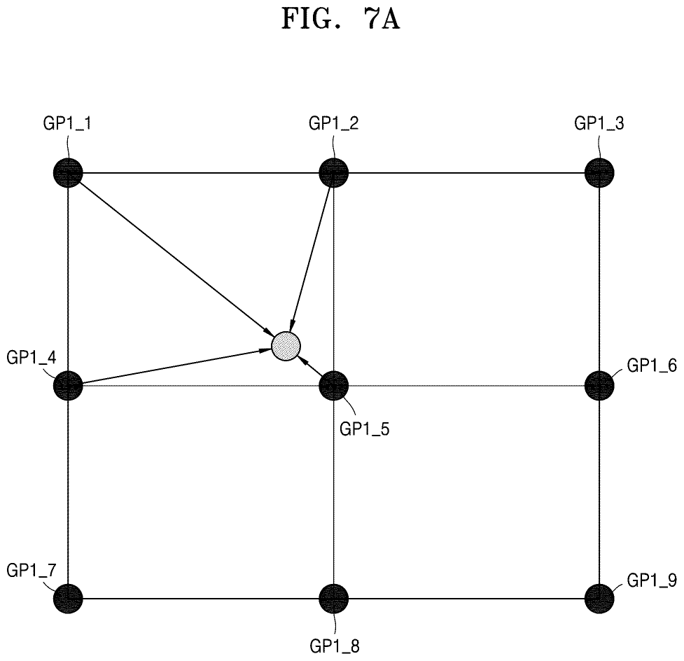

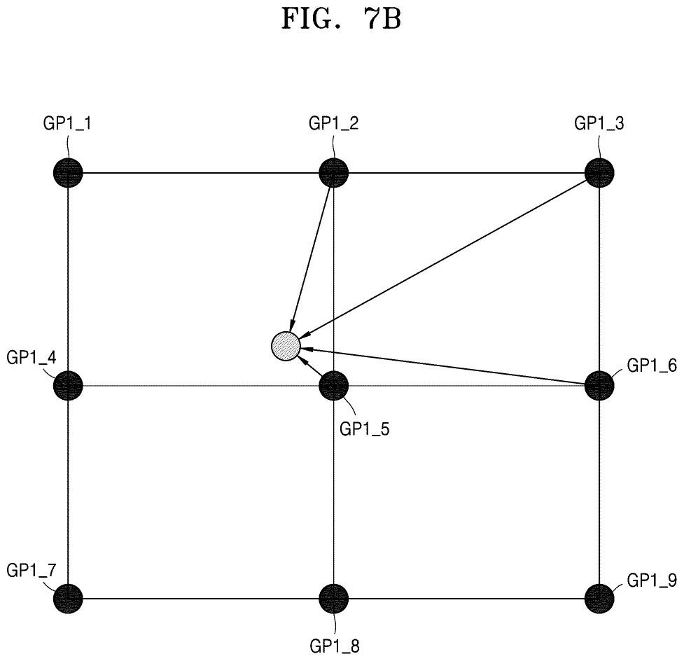

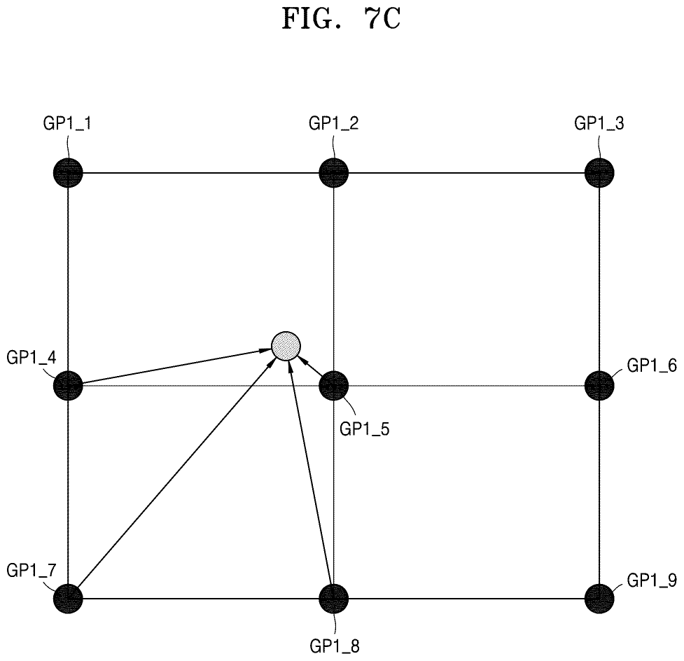

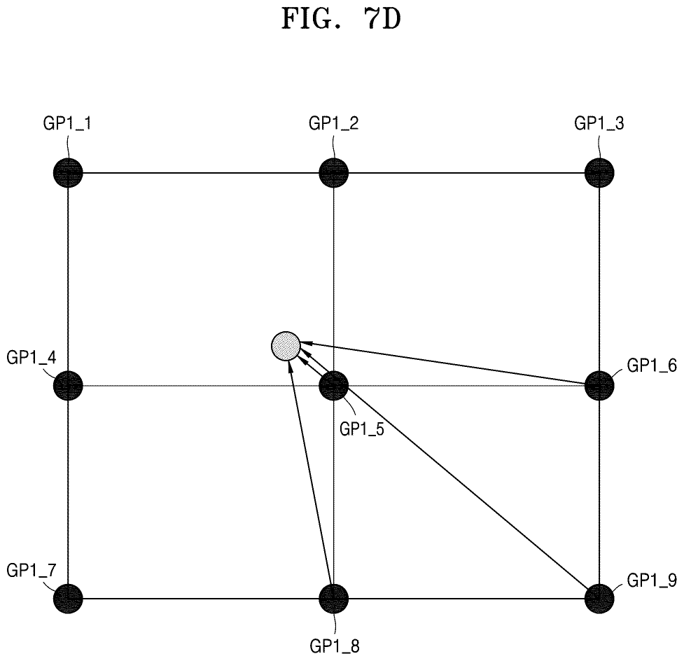

Each of the plurality of second grid point information may include position information of each of the plurality of second grid points and energy information of each of the plurality of second grid points. At this time, an example in which each of the plurality of first network switches 120_1_1 to 120_1_4 generates each of the plurality of second grid point information will be described below with reference to FIGS. 7A to 7D.

After one or more of the plurality of proximity forces are calculated in each of the plurality of memory devices 110_1 to 110_16 at a first time point, each of the plurality of first network switches 120_1_1 to 120_1_4 may generate each of the plurality of second grid point information at the first time point by calculating the plurality of first grid forces at the first time point based on at least some of the plurality of first grid point information at the second time point, which is a time point after a reference time has elapsed from the first time point. That is, after the plurality of proximity forces are calculated by each of the plurality of memory devices (110_1 to 110_16), each of the plurality of first network switches 120_1_1 to 120_1_4 may generate each of the plurality of second grid point information by calculating the plurality of first grid forces.

Each of the plurality of first network switches 120_1_1 to 120_1_4 may generate each of the plurality of second grid point information by calculating the plurality of first grid forces at the first time point based on at least some of the plurality of first grid point information when at least one of the plurality of proximity forces at the second time point is calculated by each of the plurality of memory devices 110_1 to 110_16.

In this way, by calculating the plurality of proximity forces in each of the plurality of memory devices 110_1 to 110_16 and simultaneously calculating the plurality of first grid forces in each of the plurality of first network switches 120_1_1 to 120_1_4, the total time required for calculating the reference particle force may be reduced.

Each of the plurality of first network switches 120_1_1 to 120_1_4 may transmit the generated plurality of second grid point information to the second network switch 120_2. In addition, each of the plurality of first network switches 120_1_1 to 120_1_4 may transmit the calculated plurality of first grid forces to the host device 130 via the second network switch 120_2.

The second network switch 120_2 may receive the plurality of second grid point information. The second network switch 120_2 may generate third grid point information by calculating a plurality of second grid forces, which are forces between a plurality of second grid points corresponding to a plurality of second grid point information and a reference particle. When calculating a plurality of grid forces using the memoization method, the second network switch 120_2 may store a second lookup table that stores the results of the calculation used for calculating a plurality of second grid forces and may calculate a plurality of first grid forces using the second lookup table.

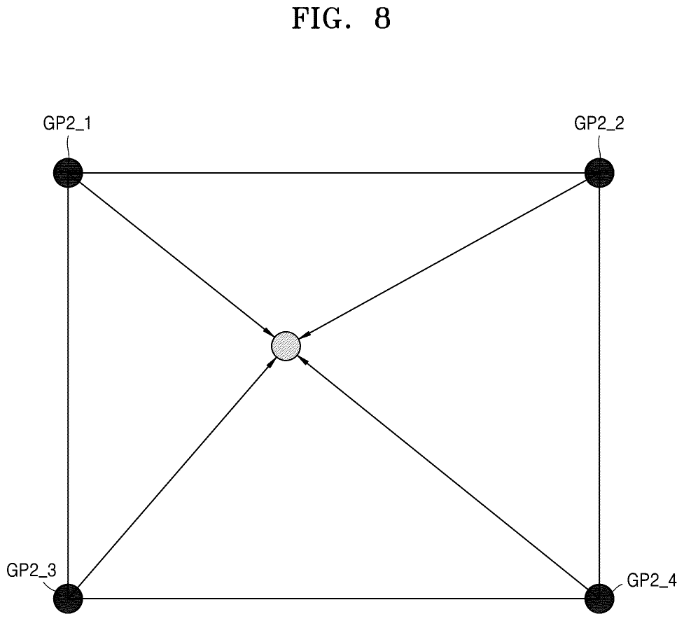

The third grid point information may include position information of the third grid point and energy information of the third grid point. At this time, an example in which the second network switch 120_2 generates the third grid point information will be described below with reference to FIG. 8.

The second network switch 120_2 may generate the third grid point information of the first time point by calculating the second grid forces of the first time point based on the plurality of second grid point information of the first time point at a third time point, which is a time point after a reference time has elapsed from the second time point by each of the plurality of memory devices 110_1 to 110_16. That is, the second network switch 120_2 may generate the third grid point information by calculating the plurality of second grid forces after the plurality of proximity forces are calculated in each of the plurality of memory devices 110_1 to 110_16, the plurality of first grid forces are calculated in the plurality of first network switches 120_1_1 to 120_1_4, and the reference time has elapsed.

In this way, by sequentially performing the calculations, like the calculation of the plurality of second grid forces at the second network switch 120_2 at a time point after calculating the plurality of first grid forces at each of the plurality of first network switches 120_1_1 to 120_1_4, the total time required for calculating the reference particle force may be reduced.

The second network switch 120_2 may transmit the generated third grid point information to the host device 130. In addition, the second network switch 120_2 may transmit the calculated plurality of second grid forces to the host device 130, and the plurality of second grid forces may be transmitted by being included in the third grid point information.

The host device 130 may receive the third grid point information. The host device 130 may calculate the reference particle force based on the third grid point information. For example, the host device 130 may calculate the reference particle force by adding up the received plurality of proximity forces, the plurality of first grid forces, and the plurality of second grid forces.

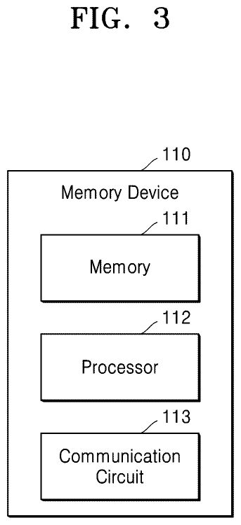

FIG. 3 is a block diagram illustrating a memory device according to an embodiment.

Referring to FIG. 3, the memory device 110 according to an embodiment may include a memory 111, a processor 112, and a communication circuit 113. The memory device 110 may be any one of the plurality of memory devices 110_1 to 110_16 illustrated in FIG. 2.

The memory 111 may store particle data. The particle data may include at least one of position information of particles, velocity information of the particles, energy information of the particles, mass information of the particles, and a list of nearby neighbors of the particles as information about the reference particle and the plurality of associated particles. However, the information included in the particle data is not limited to thereto, and may include more types of information, such as information specialized according to technique, such as quantum mechanics, machine learning, etc., depending on the calculation method using the particle data.

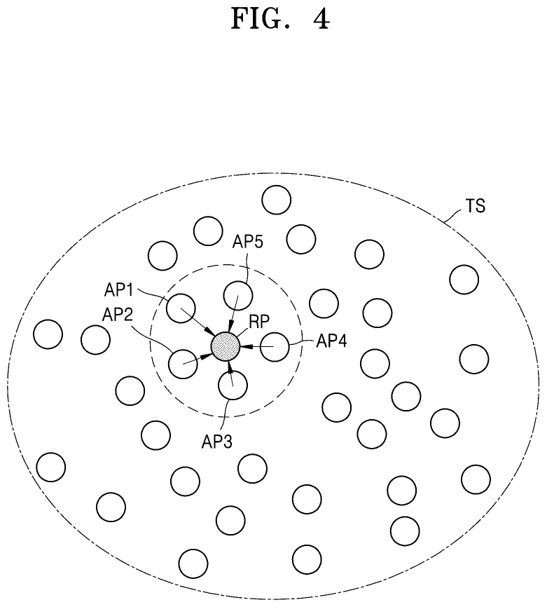

The processor 112 may calculate one or more of the plurality of proximity forces, which are forces between the reference particle and the plurality of proximity associated particles located within a reference distance from the reference particle among the plurality of associated particles. The processor 112 may include an Application Specific Integrated Circuit (ASIC), a Field Programmable Gate Array (FPGA), a GPU, etc., which may accelerate the calculation speed of the plurality of proximity forces. A method of calculating the proximity force may be described in more detail with reference to FIG. 4.

FIG. 4 is a diagram for explaining a method of calculating a plurality of proximity forces in a memory device according to an embodiment.

Referring to FIG. 4, a diagram showing a target material TS and a plurality of associated particles included in the target material TS may be confirmed. The target material TS may have a region as indicated by a dashed line. The target material TS may include a plurality of associated particles as indicated by a solid circle inside.

Any one of the plurality of associated particles included in the target material TS may be set as a reference particle RP. In the embodiment of FIG. 4, a particle, an interior of which is filled with gray shading, may be the reference particle RP.

A circle having a reference distance as a radius centered on the reference particle RP is expressed as a dotted line, and particles located within the reference distance from the reference particle RP may be set as a plurality of proximity associated particles AP1 to AP5. In the embodiment of FIG. 4, only five proximity associated particles AP1 to AP5 are illustrated, but this is due to the limitations of the drawing and in reality, a much larger number of proximity associated particles may exist.

The processor 112 may calculate a proximity force between the reference particle RP and one of the proximity associated particles AP1 to AP5. At this time, the proximity force may be forces as illustrated by solid arrows in FIG. 4 and may be usually calculated using a formula for calculating forces between particles. For example, the processor 112 included in the k^{th} memory device 110_k may calculate a proximity force between the reference particle RP and the k^{th} proximity associated particle APK. At this time, if the number of the proximity associated particles AP1 to AP5 is greater than the number of memory devices 110_1 to 110_16, two or more proximity forces may be calculated within one memory device 110.

Re-referring to FIG. 3, the processor 112 may accelerate the computation of the plurality of proximity forces in various ways. For example, the processor 112 may perform the same number of computations regardless of the number of the plurality of proximity associated particles based on machine learning.

The processor 112 may generate grid point information by replacing the plurality of associated particles with grid points based on particle data. The processor 112 may generate grid point information based on energy information of the plurality of associated particles included in the particle data. The grid point information may include grid point position information and grid point energy information. At this time, the grid point information generated by the plurality of memory devices 110_1 to 110_16 may be the first grid point information. The method of generating the grid point information may be described in more detail with reference to FIG. 5.

FIG. 5 is a diagram for explaining grid point information generated by a memory device according to an embodiment.

Referring to FIG. 5, a diagram in which a grid is added by substituting a plurality of associated particles included in the target material TS in the diagram of FIG. 4 may be confirmed.

The processor 112 may substitute the target material TS with a square grid as illustrated in FIG. 5. At this time, the processor 112 may substitute a plurality of associated particles included in the target material TS with a plurality of grid points GP1_1 to GP1_9. For example, the processor 112 may replace a plurality of associated particles included in the target material TS with a plurality of grid points GP1_1 to GP1_9, interiors of which are filled with black shading. At this time, in the embodiment of FIG. 5, some of the vertices of the grid are replaced with a plurality of grid points GP1_1 to GP1_9, but the embodiment is not limited thereto.

At this time, the processor 112 may determine the position information of the plurality of grid points GP1_1 to GP1_9 by considering a size and position of the target material TS. In addition, the processor 112 may determine the energy information of the plurality of grid points GP1_1 to GP1_9 included in the grid point information based on the energy information of the plurality of associated particles included in the particle data. At this time, the processor 112 may determine the position information and energy information of the plurality of grid points GP1_1 to GP1_9 by performing an interpolation operation. The processor 112 may determine the energy information of the plurality of grid points GP1_1 to GP1_9 so that the total energy of the plurality of associated particles included in the target material TS is evenly distributed to the plurality of grid points GP1_1 to GP1_9. The processor 112 may generate grid point information including the position information of the determined grid point and the energy information of the grid point as described above.

The processor 112 may generate information on any one of the plurality of grid points GP1_1 to GP1_9 as the first grid point information. For example, the processor 112 may generate information on the grid point GP1_1 as the first grid point information in the first memory device 110_1.

Re-referring to FIG. 3, the operation of generating grid point information through the processor 112 as described above through FIG. 5 may be performed during the initial operation of the data processing system 100.

The communication circuit 113 may transmit one or more proximity forces and grid point information calculated by the processor 112 to any one of the plurality of first network switches 120_1_1 to 120_1_4.

The communication circuit 113 may transmit information about any one of the plurality of grid points GP1_1 to GP1_9 as first grid point information to any one of the plurality of first network switches 120_1_1 to 120_1_4. For example, the communication circuit 113 included in the first memory device 110_1 may transmit information about the grid point GP1_1 as first grid point information to a 1-1 network switch 120_1_1.

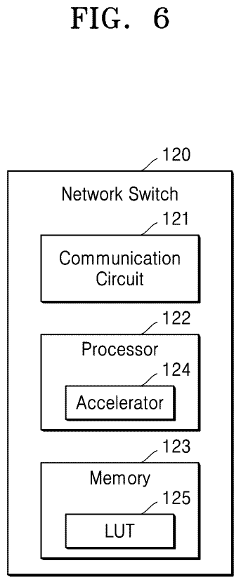

FIG. 6 is a block diagram illustrating a network switch 120 according to an embodiment.

Referring to FIG. 6, the network switch 120 according to an embodiment may include a communication circuit 121, a processor 122, and a memory 123. The network switch 120 may be any one of the plurality of network switches 120_1_1 to 120_1 4, 120_2 illustrated in FIG. 2.

The communication circuit 121 may receive a plurality of input grid point information from the plurality of memory devices 110_1 to 110_16 or the plurality of lower-level network switches. For example, the communication circuit 121 included in the plurality of first network switches 120_1_1 to 120_1_4 may receive a plurality of first grid point information from the plurality of memory devices 110_1 to 110_16 as the plurality of input grid point information. The communication circuit 121 included in the second network switch 120_2 may receive a plurality of second grid point information from the plurality of first network switches 120_1_1 to 120_1_4, which are a plurality of lower-level network switches, as the plurality of input grid point information.

The processor 122 may generate output grid point information by calculating a plurality of grid forces, which are forces between each of the plurality of input grid points corresponding to the plurality of input grid point information and the reference particle.

For example, the processor 122 included in the plurality of first network switches 120_1_1 to 120_1_4 may generate second grid point information as output grid point information based on the plurality of first grid point information. The communication circuit 121 included in the second network switch 120_2 may generate third grid point information as output grid point information based on the plurality of second grid point information. The processor 122 may include an ASIC, an FPGA, a GPU, etc., which may accelerate the calculation speed of the plurality of grid forces. The method of generating output grid point information may be described in more detail with reference to FIGS. 7A to 8.

FIGS. 7A to 7D are diagrams for explaining a method of calculating a plurality of first grid forces in each of a plurality of first network switches according to an embodiment.

First, referring to FIG. 7A, a diagram showing a plurality of first grid forces calculated by the processor 122 of the 1-1 network switch 120_1_1 may be confirmed. The processor 122 of the 1-1 network switch 120_1_1 may receive information about grid points GP1_1, GP1_2, GP1_4, and GP1_5 as a plurality of input grid point information. The processor 122 of the 1-1 network switch 120_1_1 may calculate a plurality of first grid forces between each of the grid points GP1_1, GP1_2, GP1_4, and GP1_5 and the reference particle. At this time, the plurality of first grid forces calculated by the processor 122 of the 1-1 network switch 120_1_1 may be represented by solid arrows.

The processors 122 of the 1-1 network switch 120_1_1 may generate output grid point information by determining a position corresponding to a destination point of a vector resulting from the sum of the plurality of first grid forces as position information of an output grid point and determining a size of a vector resulting from the sum of the plurality of first grid forces as energy information of the output grid point.

Referring to FIG. 7B, a diagram showing multiple first grid forces calculated by the processor 122 of the 1-2 network switch 120_1_2 may be confirmed. The processor 122 of the 1-2 network switch 120_1_2 may receive information about grid points GP1_2, GP1_3, GP1_5, and GP1_6 as a plurality of input grid point information. The processor 122 of the 1-2 network switch 120_1_2 may calculate the plurality of first grid forces between each of the grid points GP1_2, GP1_3, GP1_5, and GP1_6 and the reference particle. At this time, the plurality of first grid forces calculated by the processor (122) of the 1-2 network switch (120_1_2) may be the same as the solid arrows. The method of generating output grid point information based on the plurality of first grid forces may be the same as described above with reference to FIG. 7A.

Referring to FIG. 7C, a diagram showing the plurality of first grid forces calculated by the processor 122 of the 1-3 network switch 120_1_3 may be confirmed. The processor 122 of the 1-3 network switch 120_1_3 may receive information about grid points GP1_4, GP1_5, GP1_7, and GP1_8 as a plurality of input grid point information. The processor 122 of the 1-3 network switch 120_1_3 may calculate a plurality of first grid forces between each of the grid points GP1_4, GP1_5, GP1_7, and GP1_8 and the reference particle. At this time, the plurality of first grid forces calculated by the processor 122 of the 1-3 network switch 120_1_3 may be the same as the solid arrows. The method of generating output grid point information based on the plurality of first grid forces may be the same as described above with reference to FIG. 7A.

Referring to FIG. 7D, a diagram showing the plurality of first grid forces calculated by the processor 122 of the 1-4 network switch 120_1_4 may be confirmed. The processor 122 of the 1-4 network switch 120_1_4 may receive information about the grid points GP1_5, GP1_6, GP1_8, and GP1_9) as a plurality of input grid point information. The processor 122 of the 1-4 network switch 120_1_4 may calculate the plurality of first grid forces between each of the grid points GP1_5, GP1_6, GP1_8, and GP1_9 and the reference particle. At this time, the plurality of first grid forces calculated by the processor 122 of the 1-4 network switch 120_1_4 may be the same as the solid arrows. The method of generating output grid point information based on the multiple first grid forces may be the same as described above with reference to FIG. 7A.

FIG. 8 is a diagram for explaining a method of calculating a second grid force in a second network switch according to an embodiment.

Referring to FIG. 8, a diagram showing a plurality of second grid forces calculated by a processor 122 of a second network switch 120_2 may be confirmed. The processor 122 of the second network switch 120_2 may receive information about grid points GP2_1, GP2_2, GP2_3, and GP2_4 as a plurality of input grid point information. The processor 122 of the second network switch 120_2 may calculate a plurality of second grid forces between each of the grid points GP2_1, GP2_2, GP2_3, and GP2_4 and a reference particle. At this time, the plurality of second grid forces calculated by the processor 122 of the second network switch 120_2 may be the same as a solid arrow.

The processors 122 of the second network switch 120_2 may generate the output grid point information by determining a position corresponding to the destination point of a vector resulting from the sum of the plurality of second grid forces as position information of the output grid point and determining a size of the vector resulting from the sum of the plurality of second grid forces as energy information of the output grid point.

Re-referring to FIG. 6, the processor 122 may accelerate the calculation of the plurality of grid forces through various methods. For example, the processor 122 may accelerate the calculation of the plurality of grid forces by using a memoization method. The memoization method may be a method of accelerating the calculation by storing the previous calculation result in the memory 123 so as not to repeat the same calculation and reading the calculation result from the memory 123 without a separate calculation when the same calculation needs to be performed.

The processor 122 may include an accelerator 124. The accelerator 124 may have a structure configured to accelerate the calculation speed of the plurality of grid forces. When performing calculations of the plurality of grid forces using the memoization method, the accelerator 124 may directly access a lookup table 125 in the memory 123 as described below when necessary. That is, when performing calculations of the plurality of grid forces using the memoization method, the processor 122 may use the lookup table 125 via the accelerator 124 to calculate the plurality of grid forces.

When performing calculations of the plurality of grid forces using the memoization method, the memory 123 may store the lookup table 125 that stores the results of the calculations used in calculating the plurality of grid forces. At this time, the processor 122 may calculate the plurality of grid forces using the lookup table 125. For example, the plurality of grid forces may be calculated using the following Equation 1.

$${F}_{j}^{MM} = {\sum }_{i = 1}^{N} {r}_{ij} \frac{{q}_{i}}{{\vert {r}_{ij} \vert}^{3}} {q}_{j} \qquad [ Equation 1 ]$$

In the above Equation 1, Cambria Math may represent the total sum of the plurality of grid forces between the reference particle j and N grid points, N may represent the total number of grid points, i may represent an index of the grid point, j may represent an index of the reference particle, rᵢⱼ may represent a distance between the reference particle j and grid point i, qᵢ may represent a charge of the grid point i, and qⱼ may represent a charge of the reference particle. In Equation 1, rᵢⱼ may be calculated by using the coordinates of the input grid point included in the position information of the input grid point included in the input grid point information and the coordinates of the reference particle. In addition, qi may be calculated based on the energy information of the input grid point included in the input grid point information.

When calculating the grid forces based on the Equation 1, a cubic operation included in the denominator of the right side may take a relatively long time. At this time, each of the plurality of first network switches 120_1_1 to 120_1_4 may store a first lookup table that stores results of the cubic operation used in the calculation of the plurality of first grid forces and may calculate the plurality of first grid forces using the first lookup table. In addition, the second network switch 120_2 may store a second lookup table that stores results of the cubic operation used in the calculation of the plurality of second grid forces and may calculate the plurality of first grid forces using the second lookup table.

In this way, by calculating the plurality of grid forces using the lookup table 125 that stores the results of the relatively time-consuming calculation stored in the memory 123, the time required for the calculation may be reduced, and the time required for the entire grid force calculation may be reduced.

The communication circuit 121 may transmit information on any one of the plurality of grid points as output grid point information to any one of the upper-level network devices and the host device 130. For example, the communication circuit 121 included in the plurality of first network switches 120_1_1 to 120_1_4 may transmit information about the plurality of second grid points GP2_1 to GP2_4 as second grid point information to the second network switch 120_2. The communication circuit 121 included in the second network switch 120_2 may transmit second grid point information to the host device 130.

According to the memory device of the disclosure, the initial operation of a reference particle force operation may be performed quickly by generating grid point information and performing operations on some of the plurality of proximity forces.

In addition, according to the data processing system of the disclosure, the reference particle force operation may be performed at a faster speed by calculating the proximity force in the memory device and calculating the grid force in the network switch.

As described above, the embodiments have been disclosed in the drawings and specifications. In the present specification, the example embodiments are described by using some specific terms, but the terms used are for the purpose of describing the disclosure only and are not intended to be limiting of meanings or the technical scope described in the claims. Therefore, it will be understood by those skilled in the art that various changes in form and details may be made therein without departing from the spirit and scope of the inventive concept as defined by the appended claims. Accordingly, the scope of the invention is defined not by the detailed description of the invention but by the appended claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A memory device comprising:
a memory storing particle data about a reference particle and a plurality of associated particles; and
a processor configured to:
generate grid point information by replacing the plurality of associated particles with grid points based on the particle data; and
calculate at least one of a plurality of proximity forces defined as forces between a plurality of adjacent associated particles located within a reference distance from the reference particle among the plurality of associated particles and the reference particle.

2. The memory device of claim 1, wherein
the particle data includes at least one of position information of particles included in a target material, velocity information of the particles, energy information of the particles, mass information of the particles, and a list of nearby neighbors of the particles.

3. The memory device of claim 1, wherein
the processor is further configured to generate the grid point information based on the energy information of the plurality of associated particles included in the particle data.

4. The memory device of claim 1, wherein
the grid point information includes location information of the grid point and energy information of the grid point.

5. The memory device of claim 1, further comprising
a communication circuit configured to transmit one or more proximity forces calculated by the processor and the grid point information to one of a plurality of network switches.

6. A network switch comprising:
a communication circuit configured to receive a plurality of input grid point information from a plurality of memory devices or a plurality of lower-level network switches; and
a processor configured to generate output grid point information by calculating a plurality of grid forces defined as forces between each of a plurality of input grid points corresponding to the plurality of input grid point information and a reference particle.

7. The network switch of claim 6, wherein
the input grid point information includes location information of the input grid point and energy information of the input grid point.

8. The network switch of claim 6, further comprising
a memory storing a lookup table for storing results of operations used in the calculation of the plurality of grid forces,
wherein the processor includes an accelerator configured to calculate the plurality of grid forces using the lookup table.

9. The network switch of claim 6, wherein
the output grid point information includes position information of the output grid point determined based on the plurality of grid forces, and energy information of the output grid point.

10. The network switch of claim 6, wherein
the communication circuit is further configured to transmit the output grid point information to either a higher-level network device or a host device.

11. A data processing system configured to calculate a reference particle force defined as a force between a reference particle and a plurality of associated particle, and perform communication using a Computer Express Link (CXL), the data processing system comprising:
a plurality of memory devices configured to:
generate a plurality of first grid point information by replacing the plurality of associated particles with first grid points based on particle data about a reference particle and a plurality of associated particles; and
calculate at least one of a plurality of proximity forces defined as forces between a plurality of adjacent associated particles located within a reference distance from the reference particle among the plurality of associated particles and a reference particle;
a plurality of first network switches configured to:
receive at least some of the plurality of first grid point information; and
generate the plurality of second grid point information by calculating a plurality of first grid forces defined as forces between each of a plurality of first grid points corresponding to at least some of the plurality of first grid point information and a reference particle;
a second network switch configured to:
receive the plurality of second grid point information; and
generate third grid point information by calculating a plurality of second grid forces defined as forces between each of the plurality of second grid points corresponding to the plurality of second grid point information and a reference particle; and
a host device configured to receive the third grid point information and calculate the reference particle force based on the third grid point information.

12. The data processing system of claim 11, wherein
each of the plurality of first grid point information includes position information of each of the plurality of first grid points and energy information of each of the plurality of first grid points,
each of the plurality of second grid point information includes position information of each of the plurality of second grid points and energy information of each of the plurality of second grid points, and
the third grid point information includes position information of the third grid point and energy information of the third grid point.

13. The data processing system of claim 11, wherein
the plurality of first network switches
store a first lookup table that stores results of operations used in the calculation of the plurality of first grid forces, and
are configured to calculate the plurality of first grid forces using the first lookup table,
wherein the second network switch is configured to
store a second lookup table that stores results of operations used in the calculation of the plurality of second grid forces, and
calculate the plurality of second grid forces using the second lookup table.

14. The data processing system of claim 11, wherein
each of the plurality of first network switches is configured to:
generate the plurality of second grid point information of a first time point by calculating the plurality of first grid forces of the first time point, based on at least some of the plurality of first grid point information at a second time point
wherein the second time point is defined as a time point after a reference time has elapsed from the first time point,
wherein the plurality of second grid point information of a first time point is generated after at least one of the plurality of proximity forces of the first time point is calculated at the first time point by each of the plurality of memory devices.

15. The data processing system of claim 11, wherein
the second network switch is further configured to:
generate the third grid point information of the first time point by calculating the plurality of second grid forces of the first time point, based on the plurality of second grid point information of the first time point at a third time point,
wherein the third time point is a time point after the reference time has elapsed from the second time point.

16. The data processing system of claim 14, wherein
each of the plurality of first network switches is further configured to:
generate the plurality of second grid point information of the first time point by calculating the plurality of first grid forces of the first time, based on at least some of the plurality of first grid point information,
wherein the plurality of second grid point information of the first time point is generated when at least one of the plurality of proximity forces at the second time is calculated by each of the plurality of memory devices.
